(19) United States
(12) Patent Application Publication
(10) Pub. No.: US 2020/0219622 A1
(43) Pub. Date: Jul. 9, 2020

(54) SYSTEM AND METHODS FOR ENHANCED RISK ADJUSTMENT FACTOR PREDICTION

(71) Applicant: BASEHEALTH, INC., Sunnyvale, CA (US)

(72) Inventors: Hadi Zarkoob, Sunnyvale, CA (US); Prakash Menon, Sunnyvale, CA (US); Prasanna Desikan, Sunnyvale, CA (US); Hossein Fakhrai-Rad, Sunnyvale, CA (US); Harshna Kapashi, Sunnyvale, CA (US)

(21) Appl. No.: 16/556,010

(22) Filed: Aug. 29, 2019

Related U.S. Application Data
(60) Provisional application No. 62/788,528, filed on Jan. 4, 2019.

(51) Int. Cl. G16H 50/30; G06Q 40/08; G16H 50/20

(52) U.S. Cl. CPC G16H 50/30; G16H 50/20; G06Q 40/08

(57) ABSTRACT

An enhanced risk management method is provided in which a diverse set of inputs, such as demographic variables, risk adjustment factors (RAF) of previous years, and claims of previous years, are used in the training of a prediction model configured to predict both a standard RAF based on the assumption that a healthcare system in question continues its current, possibly suboptimal, operations, and an improved RAF based on an idealized workflow in which all of a member's Hierarchical Condition Category (HCC) codes are captured appropriately at the earliest time possible.

### CROSS-REFERENCE TO RELATED APPLICATION

This application claims the benefit under 35 USC 119(e) of U.S. Provisional Patent Application No. 62/788,528, filed Jan. 4, 2019 in the United States Patent and Trademark Office, the disclosure of which is hereby incorporated by reference in its entirety.

### BACKGROUND

### 1. Field

Apparatuses and methods consistent with exemplary embodiments relate to identifying patients at a high risk for healthcare events in the Medicare beneficiary population, and more specifically, to determining appropriate payments to be received from The Centers for Medicare & Medicaid Services (CMS) with respect to such patients.

### 2. Description of the Related Art

There are many risk adjustment models in the health insurance space for Medicare and Medicaid programs such as Medicare Advantage (the CMS-Hierarchical Condition Category (HCC) model), the Medicaid Managed Care and Healthcare Insurance Market place (Health and Human Services-Condition Categories (HHS-CC) model), with the primary purpose to ensure the payment to the plans are accurate and appropriate as per the risk of the beneficiaries enrolled in a particular time period. Among these various models, the CMS-HCC model is a prospective model which ensures a plan is paid as per the expected risk of the population that it is responsible for. This model is designed for the Medicare Advantage (MA) populations. MA has seen steady increase in enrollment from 18% of Medicare enrollees in 1999 to 33% of Medicare enrollees by 2017 (see Gretchen Jacobson, Anthony Damico, Tricia Neuman, and Marsha Gold, Medicare Advantage 2017 Spotlight: Enrollment Market Update https://www.kff.org/medicare/issue-brief/medicare-advantage-2017-spotlight-enrollment-market-update, the contents of which are hereby incorporated by reference).

A Risk Adjustment Factor (RAF) is the risk score computed by the CMS-HCC risk adjustment model. In essence, the RAF is the score that is used for Medicare patients to determine an amount that CMS pays the insurance company in a particular year for those enrolled in MA. RAF scores may be prospective in nature. Data from a previous year of service may be used to predict the expected risk and hence the prospective payment in a current year. There may be at least two key factors in understanding the payments from the RAF scores. First, may be how the score itself is computed based on key inputs. Second, may be a timeline of submission and payments.

Timeline: This may be illustrated with an example in which the prospective payments are paid for the plan year, 2017 based on the expected risk determined by the services rendered in 2016.

FIG. 1 illustrates an exemplary timeline of submissions and the eventual payments by CMS. For plan year 2017, the first submission occurs in September 2016 based on dates of service from July 2015 to June 2016. The claims from these dates of services determine the initial prospective payments from CMS on a per-member, per-month (PMPM) basis from January to June. The second submission occurs in March 2017 and includes all appropriate claims from January 2016 to December 2016. Any corrections or adjustments for the earlier claims may be made in a lump-sum amount in July or August of 2017, and the adjusted prospective payment based on the new data is made from July 2017 to December 2017. A final submission occurs in January 2018 and includes any final corrections, and any adjustments are finally made in August 2018.

Current frameworks for determining RAF scores and processing appropriate payments falls short due, at least in part, with its limited input of past services. These past services, alone, often does not accurately predict future possible healthcare events and adequate payment.

### SUMMARY

Exemplary embodiments may address at least the above problems and/or disadvantages and other disadvantages not described above. Also, exemplary embodiments are not required to overcome the disadvantages described above, and may not overcome any of the problems described above.

One or more exemplary embodiments may provide identification of patients at a high risk for healthcare events in the Medicare beneficiary population. This risk identification at appropriate times may help manage the overall health of the individual and/or the population, receive appropriate payments from CMS to manage risk, and reduce healthcare costs while improving quality measures.

According to an aspect of an example embodiment, an enhanced risk management method includes: calculating a standard risk adjustment factor (RAF_{NNR}) based on current operations; calculating an improved RAF_{ANR} based on improved operations, different from the current operations by applying a formula: RAF_{ANR}(N)=max{RAF_{NNR}(N), RAF_{NNR}(N+1), . . . , RAF_{NNR}(N+M), wherein N is a current year, and M is a non-zero integer.

The method may further include: training a model to predict a desirable output at a year N−2, based on data from years {N−3, N−4, . . . N−k−2} wherein N is a non-zero integer and 1≤k≤N−3 denotes the numbers of years of history used for training; predicting an output at a year N−1, the output being designated as \hat{p}_{N-1}, by applying the model to data in years {N−2, N−3, . . . N−k−1}; calculating b_{N-1}=p_{N-1}−\hat{p}_{N-1}, wherein b_{N-1} is bias, fitting a smooth function, designated as bias function B_{N-1}, that models b_{N-1} as a function of \hat{p}_{N-1}.

The method may further include: training a model to predict a desirable output at a year N−1, based on data from years {N−2, N−3, . . . N−k−1}; predicting an output at a year N, the output being designated as \hat{p}_{N}, by applying the model to data in years {N−1, N−3, . . . N−k−2}; applying a bias function B_{N-1} to determine a bias-corrected estimate p̅_̅{̅N̅}̅=\hat{p}_{N}+B_{N-1}(\hat{p}_{N}).

B_{N-1} may be a smooth function that models b_{N-1} as a function of \hat{p}_{N-1}, wherein b_{N-1}=p_{N-1}−\hat{p}_{N-1}.

According to an aspect of another example embodiment, an enhanced risk management method includes: receiving inputs comprising demographic variables, risk adjustment factors (RAFs) of previous years, and claims of previous years of a group of Centers for Medicare and Medicaid Services (CMS) members; and based on the received inputs: predicting a standard RAF_{NNR} based on current operations; and predicting an improved RAF_{ANR} based on improved operations.

According to an aspect of another example embodiment, a non-transitory computer-readable medium having stored thereon software which, when executed by a processor, causes a processor to execute one or more of the methods as described above.

### BRIEF DESCRIPTION OF THE DRAWINGS

The above and/or other aspects will become apparent and more readily appreciated from the following description of example embodiments, taken in conjunction with the accompanying drawings in which:

FIG. 1 illustrates RAF Submission and Payment Timelines;

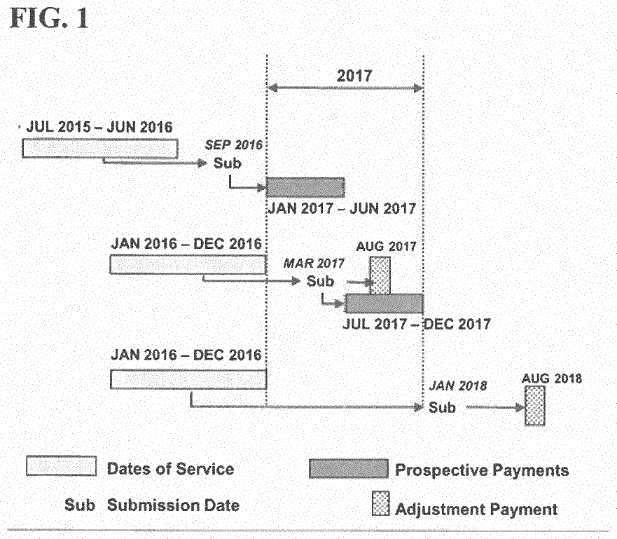

FIG. 2 is a schematic view illustrating a process of enhanced risk adjustment according to an exemplary embodiment;

FIG. 3 is a block diagram of the enhanced risk adjustment apparatus according to an exemplary embodiment;

FIG. 4 illustrates a predictive modeling infrastructure, according to an exemplary embodiment;

### DETAILED DESCRIPTION

Reference will now be made in detail to exemplary embodiments which are illustrated in the accompanying drawings, wherein like reference numerals refer to like elements throughout. In this regard, the exemplary embodiments may have different forms and may not be construed as being limited to the descriptions set forth herein.

It will be understood that the terms “include,” “including”, “comprise, and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names—this document does not intend to distinguish between components that differ in name but not function.

Matters of these example embodiments that are obvious to those of ordinary skill in the technical field to which these exemplary embodiments pertain may not be described here in detail.

Broadly speaking, RAF scores may be calculated using HCC codes, which, in turn may be functions of demographic and claims data. For a detailed calculation of an RAF score, see, for example: CMS Risk Adjustment, https://www.cms.gov/Medicare/Health-Plans/MedicareAdvtgSpecRateStats/Risk-Adjustors.html, the contents of which are hereby incorporated by reference. The final RAF scores can depend on the nature of the operational efficiency in capturing all the HCC codes. For exemplary purposes, two kinds of RAF scores may be defined.

A first kind of RAF scores are RAF scores under current operations: These are the RAF scores computed under the assumption that the healthcare system in question continues its current, possibly suboptimal, operations.

A second kind of RAF scores are RAF scores under improved operations: These are the RAF scores computed in an ideal operational workflow in which all of a member's HCC codes are captured appropriately at the earliest time possible.

Revenue Opportunities

Two revenue opportunities exist for each kind of operation (current and improved). The first one is an accelerated revenue opportunity. In this case, it some of the services rendered in the second half of 2016 were instead rendered in the first half of 2016, they could have been submitted as part of the September 2016 submission. In that way, they would have contributed to the payments made starting from January 2017. Otherwise, some of the payments are delayed until August 2017 (this delay could vary each year based on what CMS decides and announces). A second revenue opportunity is an incremental additional revenue opportunity, and is related to the payments that never get realized even with some delay. If some diagnosis codes remain unknown over the course of 2016, they would never contribute to the payments made in 2017. In the following sections we discuss ways to realize at least some of possible accelerated revenue opportunities and incremental additional revenue opportunities.

According to an exemplary embodiment, a unified integrated system and method may be provided for predicting risks for a specified population that aligns with the definition of a risk by a federal health agency. As an illustrative example, we will consider a RAF score as the risk score, Medicare Advantage as the population, and CMS as the federal health agency. All references to risk score or RAF thus refer to a RAF score or a similar risk score defined by CMS or other federal health agency.

Given the operational utility of such risk scores, exemplary desirable prediction outputs for which the system is designed may be: (1) predicted RAF under current operations, (2) predicted RAF under improved operations. As discussed above, predicted RAF scores under current operations are computed under the assumption that the healthcare system in question continues its current, possibly suboptimal, operations; and predicted RAF scores under improved operations are computed in an ideal operational workflow in which all of a member's HCC codes are captured appropriately at the earliest time possible.

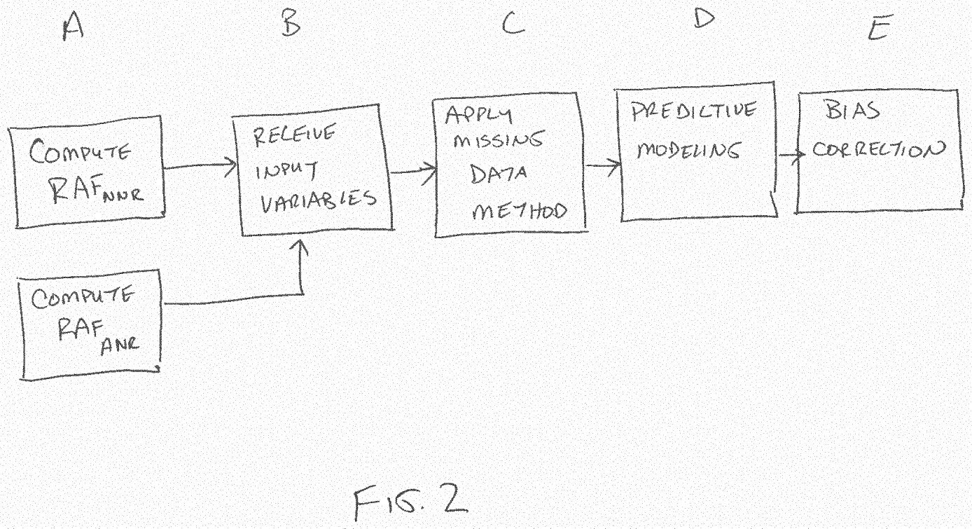

FIG. 2 is a schematic view illustrating a process of enhanced risk adjustment according to an exemplary embodiment. Referring to FIG. 2, an enhanced risk adjustment apparatus may perform the following operations to achieve the predictions as may be desired.

In operation A, the enhanced risk adjustment apparatus may calculate a measure of interest (RAF under current operations (RAF_{NNR}) or improved settings (RAF_{ANR})) in the previous years so that it can be included in a training operation, where NNR stands for normal, non-demographic, and ANR stands for achievable, non-demographic. RAF scores under current operations and RAF scores under improved operations may be calculated using appropriate methodologies, as discussed below.

In operation B, the enhanced risk adjustment apparatus may determine and receive the set of input variables (predictors) to include in model development. A variety of input variables, such as demographic variables, RAF scores from previous years, past years' claims, and the like, may be used.

In operation C, the enhanced risk adjustment apparatus may address data missing from the input variables. The enhanced risk adjustment apparatus may decide on methodology to handle missing data in the input variables, which may be present at large scales in healthcare data. An exemplary methodology may include categorizing input data into multiple data availability levels, and developing a separate model for each different level. Other missing data handling methods may be used as well. One example of missing data is observed in new members for whom past years' claims data is inherently missing.

In operation D, the enhanced risk adjustment apparatus may choose and apply an appropriate predictive modeling approach.

In operation E, the enhanced risk adjustment apparatus may apply bias correction to prevent any bias introduced due to systematic errors introduced by the methodologies.

Finally, the enhanced risk adjustment apparatus may output a candidate list of members to be inspected or otherwise managed. The enhanced risk adjustment apparatus may additionally output predictions on the RAF scores under both current and improved settings. The output may further include information used to determine monetary revenue opportunities, and/or information directed to recommendations for potential diagnoses for treatment of a particular member that may be investigated and potential interventions. Finally, the output may also include information related to potential ICD diagnosis codes. The output may be transmitted to an output device, such as a printer, external computer, display device, or other device, as would be understood by one of skill in the art.

Monetary revenue opportunities may be determined by using a methodology suggested by CMS, using the assigned base rate (from the rate book) and the determined RAF score. This enables estimation of revenue under current operations, a fully-realized revenue under improved operations, and the missed revenue. These revenues do not include rebates and bonus portions as offered by CMS.

An exemplary system, as described above, may be designed to evaluate the risks at an individual level. An ability to analyze risk at an individual-level may provide a powerful and fine-grain way to later analyze the risk adjustment programs at a population level.

One or more exemplary embodiments may enable identification of a candidate list to be inspected early in the beginning of a given year. This may be achieved by running the system described above, with respect to FIG. 2, at the beginning of the given year. The application for the system at this time of the year ensures that the healthcare organizations will have sufficient time to inspect the selected members. Note that the predicted RAF scores relate to the services that are to be provided later during the year. To this end, a machine learning model may be developed to predict an RAF score in an upcoming year based on various input elements.

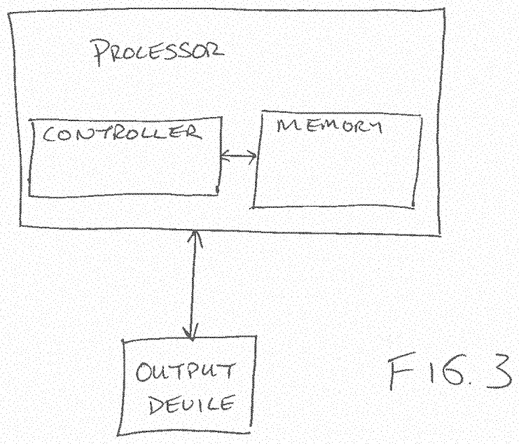

FIG. 3 is a block diagram of the enhanced risk adjustment apparatus according to an exemplary embodiment. Referring to FIG. 3, a processor may be an example of an enhanced risk adjustment apparatus. The processor includes at least a controller and a memory and is communicatively coupled to an output device, either wirelessly or via one or more hard-wired connections.

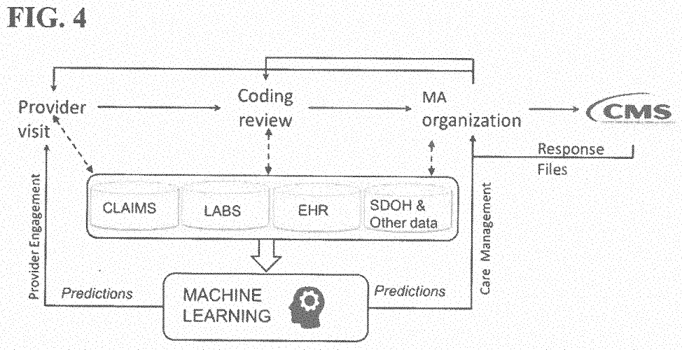

FIG. 4 illustrates how such a system may fit into the overall infrastructure of a healthcare provider. As illustrated, the system may receive a variety of relevant inputs and predict the desired outputs that may then be put into an operational flow that may end with claims submission to CMS and the payments received based on the RAF scores.

A first operation in building the model may be to have a training data that may include the desirable outputs (Operation A, as described above). The desirable outputs may include RAF scores computed under current operations and those which could have been achieved in an improved setting. The RAF under current operations can be computed in a standard methodology as defined by CMS. RAF scores may consist of two major components: (i) Demographic, and (ii) Non-demographic. The demographic part may depend on the age, gender, and ethnicity of the member. The non-demographic part may depend on the condition or diagnoses and their interactions.

Let RAF_{NNR} and RAF_{ANR} stand for normal, non-demographic RAF and Achievable, non-demographic RAF, respectively. These quantities refer to the non-demographic part of the RAF scores under the current and improved settings, respectively. Assuming RAF_{NNR} is available over years N, N+1, . . . , N+M, we suggest to estimate RAF_{ANR} at year N using the following formula:

RAF_{ANR}(N)=max{RAF_{NNR}(N),RAF_{NNR}(N+1), . . . ,RAF_{NNR}(N+M)

According to an exemplary aspect, NNRs may be down-weighted in the right-hand side of the above formula to account for the fact it may be difficult to detect/record some future ICD codes.

A potential assumption here may be that given that the health conditions in the Medicare age are usually chronic, an individual's RAF in a particular year could have been also realized in prior years, within a certain window of time, if the individual had underwent careful inspections. The reason why the above equation may rely only on the non-demographic component of RAF score may be that: (1) The non-demographic part of the RAF score may depend on the condition diagnoses. Thus, one can expect early prediction for this component. (2) The demographic component of RAF score may be known in a deterministic way even for the upcoming years. So no prediction may be needed for this component.

The next operation (operation B) may be to determine the set of input features that can contribute to the predictive ability of RAF score under current operations or improved operations. Each case can have a different set of predictors. Some exemplary features may include:

Age, Gender, Ethnicity

RAF in years N−1, N−2, . . . , N−K

Claims costs, total cost or costs under certain categories in years N−1, N−2, . . . , N−L

Set of pre-existing conditions

Lab data

Social Determinants of Health (SDOH)

Other Risk Scores

The data for these features can be directly available from standard data sources within an organization, or can be derived or obtained from external sources.

Once such data is collected, the next operation may be to handle the missing data (operation C). Missing data can be handled for many features using standard missing-data computation methodologies available. Based on the data distribution, another approach may be to develop different models for different clusters of data, as may be defined by different data availability levels. That implies, some minimum data requirements may be defined in each group and in some cases only those members that satisfy the minimum required data in each group may be assigned to that group. Once a group is formed, a group-specific model may be developed based only on the data available in that group.

For example, for new members to a Medicare Advantage (MA) plan, the past claims data and RAF scores may not be available. However, lab data and other data can be obtained from other sources. In such cases, a new model for new member may be developed that may be the most effective in its predictive ability.

A core operation (operation D) then may be to choose the right predictive model based on the input data available, the data properties and the required predictive outputs. Denoting the target prediction year by N, an exemplary embodiment for building the predictive model may include:

Age, Gender, Ethnicity

RAF in years N−1, N−2

Claims costs under the International Statistical Classification of Disease and Related Health Problems (ICD) 10 top umbrella categories in year N−1

The most extreme lab data values in the last year for certain lab measures

An additional and potentially final operation may be to handle the errors that may result from a systematic bias generated by prediction methodology (operation E). One source of bias may be due to the fact that the training operation may usually be developed based on historical data, while the test operation may need to be performed in the present. This time difference may result in some bias in the predictions.

A method to estimate and mitigate bias in the predictions may be the following. Suppose the main goal is to predict an output (RAF under current operations or under improved operations) at year N. The following may be taken to estimate a bias function that can be used to correct the prediction:

1. First, train a model that predicts the desirable output at year N−2 based on data in years N−3 and N−4.

2. Apply the model trained in the previous operation to data in years N−2 and N−3 to predict the same output at year N−1. Denote this by \hat{p}_{N-1}.

3. Compare predictions in operation 2, \hat{p}_{N-1}, with true values at year N−1, denoted by p_{N-1}. If there is any systematic error (let's say if p_{N-1} is greater than \hat{p}_{N-_1} on average by one unit, perhaps due to some time-dependent effect that could not be detected based on historical data), this signals a need for bias correction. That is because such a bias may also be present later when predicting the output at year N (the main target year). Denote bias by b_{N-1}=p_{N-1}−\hat{p}_{N-1}. Fit a smooth function (by some definition of smoothness) that models b_{N-1} as a function of \hat{p}_{N-1}. Denote this function by B_{N-1}. Later, when we get some predictions for year N, \hat{p}_{N}, we may try use this function to correct for bias in these predictions.

More specifically, the following operation may be taken to predict the scores and correct bias in additional predictions:

1. Train a model that predicts the output at year N−1, based on data in years N−2 and N−3 (main training operation).

2. Apply the trained model in operation 1 to data in years N−1 and N−2 to predict output at year N. Denote this by \hat{p}_{N}. At this operation, the output is still not corrected for bias.

3. Use function B_{N-1} learned in the previous section to correct for bias. More specifically, update predictions \hat{p}_{N} as follows: p̅_̅{̅N̅}̅=\hat{p}_{N}+B_{N-1}(\hat{p}_{N}), where p̅_̅{̅N̅}̅ denotes the bias corrected estimates.

Analysis of data across multiple years indicates bias functions do not change much over time. That implies bias functions learnt from past years data can be reasonably used to correct bias in the present year predictions.

Once RAF scores under current and optimal operations are predicted, one may use their difference to estimate missed RAF score(s) for each member. Such estimates can be directly converted to missed payments using the straightforward methodologies implemented in the CMS payment system.

A candidate chase list can be generated by selecting individuals at the top X percent of the missed revenue opportunity list, where X is a parameter controlled by user.

Integrating Other Prediction Outputs

According to an exemplary embodiment, a system may also be capable of integrating risk scores from other models as new predictive features to enhance the predictions. The system may also be capable of integrating a set of predictions that may influence RAF computations, such as the prediction of ICD codes, or any higher level of aggregation or description of clinical conditions, for individual patients (or at an aggregate level), and use such prediction outputs as predictive features in the overall prospective prediction of RAF Score.

Evaluation of a Given Candidate List

According to one or more exemplary embodiments, the following methodologies may be used to calculate missed and accelerated opportunity for any given candidate list, whether it is generated by the present method, or any other method, after the fact.

(A) Estimation of Missed Revenue Opportunity

A missed revenue opportunity can be directly estimated using calculated RAF scores (and corresponding payments) under current and optimized settings.

(B) Estimation of Accelerated Revenue Opportunity

Methodology described in this section is described with respect to the same example as discussed above. A fair and practical assumption that can be made is early inspection of individuals in the candidate list could have helped capture their diagnosis codes in the second half of 2016 earlier in the first half of 2016, given that these individuals could have been potentially known early in 2016. This assumption implies there would be no need for a delayed lump-sum payment for these individuals in August 2017. The accelerated revenue opportunity may then be evaluated by looking at the positive lump-sum adjustments made in August 2017 (available from CMS response files) for the individuals in the candidate list, and summed over the lump adjustments when they are positive.

In summary, among various Risk Adjustment models used in Medicare and Medicaid populations, the CMS-HCC model is a prospective model which is used to ensure that a plan is paid as per the expected risk of the population for which it is responsible. The score calculated by this model is called a Risk Adjustment Factor (RAF), and is applicable to Medicare Advantage populations. According to one or more exemplary embodiments a system may provide a unified framework for prospectively predicting RAF scores under current operations or operations that can more fully realize RAF score. One or more exemplary embodiments may utilize multiple novel approaches to estimate RAF scores that could have been realized in the past in an optimal operational setting. One or more exemplary embodiments may do so by, among other things, looking at claims data in the respective subsequent years. The presented unified framework is flexible in being able to consume a variety of data as well as integrating outputs from other prediction systems that can eventually be used for prospective prediction. The estimation and prediction of RAF scores may allow the system to estimate the revenue opportunity in a more fully-realized setting, as well as enabling an accelerated revenue opportunity.

The apparatuses according to exemplary embodiments may comprise a memory to store program data, a processor to execute the program data, a permanent storage unit such as a disk drive, a communication port to handle communications with external devices, and user interface devices, including a touch panel, keys, buttons, etc. When software modules or algorithms are involved, the software modules or algorithms may be stored as program instructions and/or computer readable codes executable on a processor, in a computer-readable medium. Examples of the computer readable recording medium include magnetic storage media (e.g., read-only memories (ROMs), random-access memories (RAMs), floppy disks, hard disks, etc.), and optical recording media (e.g., compact disk (CD)-ROMs, or digital versatile disks (DVDs)). The computer readable recording medium can also be distributed over network coupled computer systems so that the computer readable code is stored and executed in a distributive manner. This media can be read by the computer, stored in the memory, and executed by the processor.

Exemplary embodiments may be described in terms of functional block components and/or various processing steps. Such functional blocks may be realized by any number of hardware and/or software components configured to perform the specified functions. For example, exemplary embodiments may employ various integrated circuit (IC) components, e.g., memory elements, processing elements, logic elements, look-up tables, and the like, which may carry out a variety of functions under the control of one or more microprocessors or other control devices. Similarly, where the elements are implemented using software programming or software elements, the exemplary embodiments may be implemented with any programming or scripting language such as C, C++, Java, assembler language, or the like, with the various algorithms being implemented with any combination of data structures, objects, processes, routines or other programming elements. Functional aspects may be implemented in algorithms that are executed on one or more processors. Furthermore, the exemplary embodiments may employ any number of conventional techniques for electronics configuration, signal processing and/or control, data processing and the like. The words “mechanism,” “element,” “means,” and “configuration” are used broadly and are not limited to mechanical or physical embodiments, but may include software routines in conjunction with processors, etc.

At least one of the components, elements, modules or units represented by a block as illustrated in the drawings may be embodied as various numbers of hardware, software and/or firmware structures that execute respective functions described above, according to an exemplary embodiment. For example, at least one of these components, elements or units may use a direct circuit structure, such as a memory, a processor, a logic circuit, a look-up table, etc. that may execute the respective functions through controls of one or more microprocessors or other control apparatuses. Also, at least one of these components, elements or units may be specifically embodied by a module, a program, or a part of code, which contains one or more executable instructions for performing specified logic functions, and executed by one or more microprocessors or other control apparatuses. Also, at least one of these components, elements or units may further include or be implemented by a processor such as a central processing unit (CPU) that performs the respective functions, a microprocessor, or the like. Two or more of these components, elements or units may be combined into one single component, element or unit which performs all operations or functions of the combined two or more components, elements of units. Also, at least part of functions of at least one of these components, elements or units may be performed by another of these components, element or units. Further, although a bus is not illustrated in the above block diagrams, communication between the components, elements or units may be performed through the bus. Functional aspects of the above exemplary embodiments may be implemented in algorithms that execute on one or more processors. Furthermore, the components, elements or units represented by a block or processing steps may employ any number of related art techniques for electronics configuration, signal processing and/or control, data processing and the like.

This risk identification at appropriate times helps to manage the overall health of the individual and/or the population, receive the appropriate payments from CMS to manage their risk and helps reduce healthcare costs while improving quality measures.

The particular implementations shown and described herein are illustrative examples and are not intended to otherwise limit the scope of the disclosure in any way. For the sake of brevity, conventional electronics, control systems, software development and other functional aspects of the systems may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical apparatus.

The use of the terms “a” and “an” and “the” and similar referents in the context of describing the disclosure (especially in the context of the following claims) is to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Also, the steps of all methods described herein can be performed in any suitable order unless otherwise indicated herein or otherwise clearly contradicted by context. The disclosure is not limited to the described order of the steps. The use of any and all examples, or exemplary language (e.g., “such as”) provided herein, is intended merely to better illuminate the inventive concept and does not pose a limitation on the scope of the inventive concept unless otherwise claimed. Numerous modifications and adaptations will be readily apparent to one of ordinary skill in the art without departing from the spirit and scope.

The exemplary embodiments should be considered in descriptive sense only and not for purposes of limitation. Descriptions of features or aspects within each embodiment should typically be considered as available for other similar features or aspects in other embodiments.

Although a few embodiments have been shown and described, it would be appreciated by those skilled in the art that changes may be made in example embodiments without departing from the principles and spirit of the disclosure, the scope of which is defined in the claims and their equivalents.

What is claimed is:

1. An enhanced risk management method, the method comprising:
calculating a standard risk adjustment factor (RAF_{NNR}) based on current operations;
calculating an improved RAF_{ANR} based on improved operations, different from the current operations by applying a formula:
RAF_{ANR}(N)=max{RAF_{NNR}(N),RAF_{NNR}(N+1), . . . ,RAF_{NNR}(N+M)
wherein N is a current year, and M is a non-zero integer.

2. The method of claim 1, further comprising:
training a model to predict a desirable output at a year N−2, based on data from years N−3, N−4, . . . N−k−2, wherein N is a non-zero integer and 1≤k≤N−3;
predicting an output at a year N−1, the output being designated as \hat{p}_{N-1}, by applying the model to data in years N−2, N−3, . . . N−k−1;
calculating b_{N-1}=p_{N-1}−\hat{p}_{N-1}, wherein b_{N-1} is bias,
fitting a smooth function, designated as bias function B_{N-1}, that models b_{N-1} as a function of \hat{p}_{N-1}.

3. The method of claim 1, further comprising:
training a model to predict a desirable output at a year N−1, based on data from years N−2, N−4, . . . N−k−1;
predicting an output at a year N, the output being designated as \hat{p}_{N}, by applying the model to data in years N−1, N−3, . . . N−k; and
applying a bias function B_{N-1} to determine a bias-corrected estimate p̅_̅{̅N̅}̅=\hat{p}_{N}+B_{N-1}(\hat{p}_{N}).

4. The method of claim 3, wherein B_{N-1} is a smooth function that models b_{N-1} as a function of \hat{p}_{N-1}, wherein b_{N-1}=p_{N-1}−\hat{p}_{N-1}.

5. An enhanced risk management method, the method comprising:
receiving inputs comprising demographic variables, risk adjustment factors (RAFs) of previous years, and claims of previous years of a group of Centers for Medicare and Medicaid Services (CMS) members; and
based on the received inputs:
calculating a standard RAF_{NNR} based on current operations;
and calculating an improved RAF_{ANR} based on improved operations.

6. The method of claim 5, further comprising:
based on at least one of RAF_{NNR} and RAF_{ANR}, outputting to an output device at least one of:
a candidate list of CMS members;
recommendations of monetary revenue opportunities;
recommendations for a potential diagnosis for treatment of a CMS member;
ICD diagnosis codes.

7. A non-transitory computer-readable medium having stored thereon software which, when executed by a processor, causes a processor to execute an enhanced risk management method, the method comprising:
calculating a standard risk adjustment factor (RAF_{NNR}) based on current operations;
calculating an improved RAF_{ANR} based on improved operations, different from the current operations by applying a formula:
RAF_{ANR}(N)=max{RAF_{NNR}(N),RAF_{NNR}(N+1), . . . ,RAF_{NNR}(N+M)
wherein N is a current year, and M is a non-zero integer.

8. The non-transitory computer-readable medium of claim 7, wherein the method further comprises:
training a model to predict a desirable output at a year N−2, based on data from years N−3, N−4, . . . N−k−2, wherein N is a non-zero integer and 1≤k≤N−3;
predicting an output at a year N−1, the output being designated as \hat{p}_{N-1}, by applying the model to data in years N−2, N−3, . . . N−k;
calculating b_{N-1}=p_{N-1}−\hat{p}_{N-1}, wherein b_{N-1} is bias,
fitting a smooth function, designated as bias function B_{N-1}, that models b_{N-1} as a function of \hat{p}_{N-1}.

9. The non-transitory computer-readable medium of claim 7, wherein the method further comprises:
training a model to predict a desirable output at a year N−1, based on data from years N−2, N−3, . . . N−k;
predicting an output at a year N, the output being designated as \hat{p}_{N}, by applying the model to data in years N−1, N−2, . . . N−k; and
applying a bias function B_{N-1} to determine a bias-corrected estimate p̅_̅{̅N̅}̅=\hat{p}_{N}+B_{N-1}(\hat{p}_{N}).

10. The non-transitory computer-readable medium of claim 9, wherein the method further comprises, wherein B_{N-1} is a smooth function that models b_{N-1} as a function of \hat{p}_{N-1}, wherein b_{N-1}=p_{N-1}−\hat{p}_{N-1}.

11. A non-transitory computer-readable medium having stored thereon software which, when executed by a processor, causes a processor to execute an enhanced risk management method, the method comprising:
receiving inputs comprising demographic variables, risk adjustment factors (RAFs) of previous years, and claims of previous years of a group of Centers for Medicare and Medicaid Services (CMS) members; and
based on the received inputs:
predicting a standard RAF_{NNR} based on current operations;
and predicting an improved RAF_{ANR} based on improved operations.
